(19) United States
(12) Patent Application Publication
(10) Pub. No.: US 2023/0045097 A1
(43) Pub. Date: Feb. 9, 2023

(54) MULTI-FUNCTIONAL NANOPARTICLE TARGETED TO BREAST CANCER, PREPARATION METHOD AND USE THEREOF

(71) Applicant: WuHan University, Wuhan (CN)

(72) Inventors: Jin XIANG, Wuhan (CN); Xin WANG, Wuhan (CN); Miaomiao QI, Wuhan (CN)

(73) Assignee: WuHan University, Wuhan (CN)

(21) Appl. No.: 17/854,685

(22) Filed: Jun. 30, 2022

(30) Foreign Application Priority Data
Jul. 6, 2021 (CN) 2021107639923

(51) Int. Cl. A61K 9/51; A61P 35/00; A61K 31/704; A61K 33/00; C12P 21/02; C07K 14/47

(52) U.S. Cl. CPC A61K 9/5169; A61P 35/00; A61K 31/704; A61K 33/00; C12P 21/02; C07K 14/47; C07K 2319/50; C07K 2319/09

(57) ABSTRACT

The present disclosure relates to a multi-functional nanoparticle targeted to breast cancer, a preparation method and use thereof. The multi-functional nanoparticle includes a targeting carrier and a medicament loaded on the targeting carrier; and the targeting carrier is made from recombinant ferritin. Cell experiments verify that the multi-functional nanoparticle has better efficacy and drug release capacity for cancer cells than those of conventional ferritin as a vector. Moreover, the drug delivery system can further achieve optical imaging of tumor cells by loading quantum dots, thus playing a role in cancer diagnosis and treatment.

### CROSS-REFERENCE TO RELATED APPLICATIONS

The application claims priority to Chinese patent application No. 2021107639923, filed on Jul. 6, 2021, the entire contents of which are incorporated herein by reference.

### TECHNICAL FIELD

The present disclosure relates to the technical field of medicaments for targeted therapy of breast cancer, and in particular to a multi-functional nanoparticle targeted to breast cancer, a preparation method and use thereof.

### BACKGROUND

Ferritin is a Fe storage protein widely existing in organisms, which is responsible for maintaining Fe ion homeostasis of a body and protecting cells from oxidative damage. The ferritin superfamily can be divided into two major class groups according to size: large ferritin and small ferritin. Large ferritin ranges from 8-12 nm in size and consists of 24 subunits by themselves to form a regular octahedral symmetric cage-like structure; large ferritin exists in bacteria, archaebacteria and eukaryotes, including human ferritin, human serum albumin and bacterial ferritin. Small ferritin ranges from 4.5-9 nm in size and is assembled into a tetrahedral cage-like structure by 12 subunits; small ferritin exists in bacteria and archaebacteria.

Ferritin not only hits good biocompatibility and high stability, but also has resistances to chemical denaturation, pH change and heat resistance. Furthermore, since ferritin can form these two cage-like structures above, ferritin can serve as a good drug carrier and a biomineralized reaction vessel to synthesize ultra-small inorganic nanoparticles of Co(O)OH, Co₃O₄, Fe₂O₃, CdS, Pt, Au, FeCo, and the like in the inner cavity of the cage-like structures. Therefore, ferritin has a wide range of applications in multiple aspects such as targeted therapy, drug delivery vectors, bioimaging, biological multi-functional nanomaterials.

In addition, the self-assembly ability of ferritin can facilitate its combination with drug delivery and other functions to form a nano-structured functional complex. For example, it has been studied that ferritin fluorescent Cy5.5 molecules are conjugated or wrapped on magnetite nanoparticles, thus enabling plaque tissues to be imaged by fluorescence and magnetic resonance imaging. Due to their controllable self-assembly, nanoparticle loading capacity and easy modification, ferritin has broad potential and thus can be used as a complex multivalent structure and a platform for biomedical applications. However, directed to the solution of utilizing ferritin to wrap a diagnostic or therapeutic agent for early diagnosis and targeted therapy of breast cancer, there is no such multi-functional nanostructured complex at present.

### SUMMARY

In view of this, the objective of the present disclosure is to provide a multi-functional nanoparticle targeted to breast cancer, which is expected to solve one of the technical problems in early diagnosis and/or targeted therapy to some extent.

In a first aspect, examples of the present disclosure disclose a multi-functional nanoparticle targeted to breast cancer, including a targeting carrier and a medicament loaded on the targeting carrier; the targeting carrier is made from recombinant ferritin.

In examples of the present disclosure, the recombinant ferritin has a primary structure formed by linking a therapeutic polypeptide and MMP restriction enzyme cutting sites to ferritin.

In examples of the present disclosure, MMP restriction enzyme cutting sites are linked at both ends of the therapeutic polypeptide.

In examples of the present disclosure, the therapeutic polypeptide is an Wnt/β-catenin signaling inhibitor which is capable of preventing β-catenin from binding to LEF-1 in a nuclear region of human breast cancer cells, thereby accelerating the apoptosis of cancer cells, and reducing the growth and motility of cancer cells.

In examples of the present disclosure, the medicament includes quantum dots and adriamycin.

In a second aspect, examples of the present disclosure disclose a method for preparing the multi-functional nanoparticle disclosed in the first aspect, including the steps of preparing recombinant ferritin and loading the medicament; where,

the step of preparing the recombinant ferritin includes:

constructing a genetically engineered strain expressing the recombinant ferritin;

subjecting the genetically engineered strain to plate culture and liquid induction culture to obtain a bacterial solution containing the recombinant ferritin;

collecting bacterial cells in the bacterial solution, crushing and purifying to obtain the recombinant ferritin.

In examples of the present disclosure, the construction process of the genetically engineered strain includes: constructing an expression vector pET28a-HFn-CP1, transforming the expression vector into E. coli Rosetta (DE3) competent cells, performing streak culture on a kana+plate, and picking a positive monoclonal colony as the genetically engineered strain; where she expression vector includes nucleotide sequences for expressing the MMP restriction enzyme cutting sites, the therapeutic polypeptide and the ferritin respectively; the nucleotide sequence for expressing the MMP restriction enzyme cutting sites is shown in SEQ ID NO. 1 and the nucleotide sequence for expressing the therapeutic polypeptide is shown in SEQ ID NO. 2.

In examples of the present disclosure, the step of loading the medicament includes:

adding quantum dots and adriamycin solution to a urea solution of the recombinant ferritin for mixing, performing incubation in the dark, then dialyzing the same in a urea buffer solution to obtain a concentrate;

subjecting the concentrate to sucrose density gradient centrifugation, thus obtaining the finally purified multi-functional nanoparticle.

In examples of the present disclosure, the sucrose solution has concentration gradients of 10 w/w %, 15 w/w %, 20 w/w %, 25 w/w %, 50 w/w %, 35 w/w %, 40 w/w %, 45 w/w % and 50%, respectively.

In a third aspect, examples of the present disclosure further disclose use of the multi-functional nanoparticle disclosed in the first aspect, or the multi-functional nanoparticle prepared by the preparation method disclosed in the second aspect in the preparation of an anti-breast cancer medicament.

Compared with the prior art, the present disclosure at least has the following beneficial effects.

In examples of the present disclosure, recombinant ferritin merged with a therapeutic polypeptide directed to cancer cells is constructed. Moreover, such a unique adriamycin recombinant ferritin nanoparticle is designed and utilized as a drug delivery system which is loaded with quantum dots and adriamycin. Cell experiments verify that the drug delivery system has better efficacy and drug release capacity than those of conventional ferritin as a carrier. The results indicate that the recombinant ferritin can release the therapeutic peptide in cells better, and the adriamycin loaded can also be precisely released to achieve targeted therapy. Moreover, the loading of quantum dots can achieve optical imaging of tumor cells, thus playing a role in cancer diagnosis and treatment.

### BRIEF DESCRIPTION OF DRAWINGS

FIG. 1 is a schematic diagram showing a recombinant protein HFn-CP1 provided in the examples of the present disclosure.

FIG. 2 is a structure diagram showing an expression vector of the recombinant protein HPn-CP1 provided in the examples of the present disclosure.

FIG. 3 is a diagram showing the purification of the HFn-CP1 provided in the examples of the present disclosure; FIG. 3A is a SDS-PAGE diagram of protein primary purification-nickel column affinity chromatography; where lane M is Marker, lanes 1-2 are successively supernatant and column flowing-through effluent, lanes 3-7 are all 5 mM imidazole eluent, lanes 8-13 are all 20 mM eluent, and lanes 14-15 are 100 mM imidazole eluent; FIG. 3B is a SDS-PAGE diagram of the lanes 6-11 in FIG. 3A after being heated at 60° C. for 10 mm; FIG. 3C is a graphical result of Western Blot tor HFn-CP1.

FIG. 4 is a diagram showing the purification of a HFn wild-type protein provided in the examples of the present disclosure; FIG. 4A shows a result of SDS-PAGE; FIG. 4B is a graphical result of Western Blot.

FIG. 5 is a graphical result of sucrose gradient centrifugation of a protein of interest provided in the examples of the present disclosure; the left shows centrifugal bands of HFn-CP1(QD); the right shows the centrifugal bands of HFn-CP1(Adr+QD).

FIG. 6 is a TEM diagram of HFn(QD) nanoparticles provided by the examples of the present disclosure.

FIG. 7 is a TEM diagram of HFn-CP1(QD) nanoparticles provided by the examples of the present disclosure.

FIG. 8 is a particle size distribution characterized by DLS provided by the examples of the present disclosure; the upper graph shows the particle size distribution of HPn(QD); the lower graph shows the particle size distribution of HFn-CP1(QD).

FIG. 9 is a schematic diagram showing the inhibition of different medicaments provided in the examples of the present disclosure on cell viability; drug concentration in each group of the figure is set 0.1 mg/mL; * indicates P<0.05; ** indicates P<0.01; *** indicates P<0.001; **** indicates P<0.0001, and n=3.

FIG. 10 is a diagram showing the inhibition of different concentration gradients of HFn-CP1(Adr+QD) provided in the examples of the present disclosure on MDA-MB-468 cell viability (* indicates P<0.05, n=3).

FIG. 11 shows diagrams of different concentration gradients of Adr provided by the examples of the present disclosure and the inhibition thereof on MDA-MB-468 cell viability (* indicates P<0.05; ** indicates P<0.01; *** indicates P<0.001; **** indicates P<0.0001, and n=3).

### DETAILED DESCRIPTION OF THE EMBODIMENTS

To make the objectives, technical solutions and advantages of the present disclosure more apparent, the present disclosure will be further described in detail by reference to specific examples below. It should be understood that the specific examples described herein are merely illustrative of the present disclosure but are not construed as limiting the present disclosure.

For the purposes of early diagnosis and targeted therapy for human breast cancer, in the examples of the present disclosure, a matrix metalloproteinase is incorporated on the basis of the ferritin drug delivery system to provide targeting ability thereof as a drug delivery system.

Matrix metalloproteinases (matrix metalloproteinase, MMPs) are a family of zinc ion and calcium ion-dependent proteases. Matrix metalloproteinases can almost degrade various protein components in the extracellular matrix, destroy the histological barrier of tumor cell invasion and play a critical role in tumor invasion and metastasis. The examples of the present disclosure take advantage of the feature that the expression level of MMP enzyme in cancer tissues is higher than that in normal tissues to design a small peptide which is linked to ferritin (HFn) via MMP restriction enzyme cutting sites and has therapeutical effects on cancer. The small peptide is cleaved from the assembled nanoparticle at the tumor tissue site by the highly expressed MMP enzyme, thus exciting, the effect of targeted therapy. Meanwhile, the nanoparticle can also be loaded with quantum dots and anti-cancer drug adriamycin at the same time, thus playing the role of early diagnosis and targeted therapy.

The preparation of the multi-functional nanoparticle and properties thereof will be described below. Materials, reagents, etc. used in the following examples are commercially available, unless otherwise specified.

Multi-Functional Nanoparticle (abbreviated as HFn-CP1(Adr+QD)) and Preparation Thereof

1. Materials and Methods

1. Preparation of HFn-CP1 Recombinant Protein

1.1. Construction of pET28a-HFn-CP1 Expression Vector

A segment of modified short peptide was added on the N-terminal of wild-type human ferritin HFn. The modified short peptide consists of three parts: MMP restriction enzyme cutting sites+short peptide for cancer therapy+MMP restriction enzyme cutting sites. The cancer therapeutic polypeptide may produce inhibiting effects on the viability of breast cancer cells, and mainly consists of three parts, respectively as follows: a cell penetrating peptide, a nuclear localization peptide and a therapeutic activation region peptide.

The amino acid sequences of the MMP restriction enzyme cutting sites are shown in SEQ ID NO. 3. The MMP restriction enzyme cutting sites refer to polypeptide sequence which can be recognized and degraded by the MMPs enzyme family, namely, the characteristic degradation substrate object of the MMP enzyme.

The therapeutic polypeptide is a Wnt/β-catenin signaling inhibitor which can prevent β-catenin from binding to LEF-1 in the nuclear region of human breast cancer cells, accelerate the apoptosis of cancer cells, and reduce the growth and motility of cancer cells. The therapeutic polypeptide has an amino acid sequence as shown in SEQ ID NO. 4.

The β-catenin/Lef-1 complex is a nuclear reaction transcription factor and plays an important role in Wnt/Wingless signaling pathway. The Wnt/Wingless pathway regulates many processes of tumorigenesis, including cell growth, invasion, migration, clone formation, and human cancer xenograft. Upon dissociation of the Wnt receptor complex, β-Catenin forms a complex with adenomatous polyp tissue Escherichia coli, (APC), axin and glycogen synthase kinase 3B (GSK3B) and then undergoes ubiquitin-mediated degradation in cytoplasm. Otherwise, when Wnt binds to a receptor complex, β-catenin translocates to cell nucleus and binds to the Lef-1/Tef co-transfection factor. It is reported that the former 76 amino acids of LeF-1 are sufficient to interact with β-catenin. Moreover, it has been found through early studies that β-catenin can activate downstream target genes, including BMP4, MYC and Cyclin D1. These genes play an important role in accelerating the migration, invasion and tumorigenesis of breast cancer. Therefore, the inhibition on β-catenin/Lef-1 signaling pathway is an important direction for the development of new medicaments.

The therapeutic polypeptide selected in the examples of the present disclosure includes contiguous short peptides of transcriptional transactivator (TAT)(YGRKKRRQRRR), nuclear localization signal (NLS, RKRRK) and activation region sequence (ATDEMIPF) proteins. The transcriptional transactivator (TAT) is a penetrating peptide derived from human immunodeficiency virus. TAT can deliver proteins, deoxyribonucleic acids, ribonucleic acids and nanoparticles into cytoplasm with high efficiency in a short time. The nuclear localization signal (NLS) is to affect the binding of TCF-4/LEF-1 to the Wnt target gene due to the translocation of stable β-catenin into nucleus. The NLS may help small peptides enter into cell nucleus. Activation domain sequence (ATDEMIPF) is a Wnt/β-catenin signaling inhibitor which can block the interaction between β-catenin and Lef-1, and effectively inhibit the occurrence of tumor in vitro and in vivo.

To make the modified short peptide working normally, in this present example, MMP restriction enzyme cutting sites (PLGLWA) were designed and linked on both sides of the therapeutic polypeptide to form a CP1 structure, and then linked to HFn to form a final recombinant HFn fusion protein.

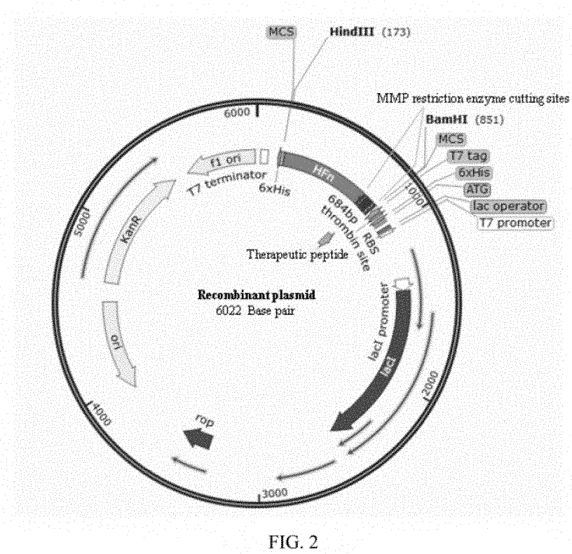

In the example of the present disclosure, based on the plasmid pET28a (+), an expression vector capable of expressing CP1 and HFn simultaneously was constructed. The specific structure of the pET28a-HFn-CP1 expression vector is shown in FIG. 2, and base sequences of HFn and CP1 are shown in Table 1. A pET28a plasmid was selected for the expression and purification of the protein of interest, and the designed plasmid was submitted to Wuhan Qingke Biotechnology Co. Ltd. for full gene synthesis.

TABLE 1
Related nucleotide sequences in the expression
vector and related amino acid sequences in the
expression protein

| Name | Sequence |
|---|---|
| Expression | CCGCTGGGTCTGTGGGCA; as |
| nucleotide sequence | shown in SEQ ID NO. 1 |
| of the MMP | |
| restriction enzyme | |
| cutting site | |
| Expression | TATGGTCGTAAAAAGCGTCGTCAGCGTC |
| nucleotide sequence | GTCGTCGTAAACGTCGTAAAGCAACCGA |
| of the therapeutic | TGAAATGATTCCGTTT, as shown |
| polypeptide | in SEQ ID NO. 2 |
| Expression | Sequence ID: NM_002032.3 |
| nucleotide sequence | |
| of the ferritin | |
| Amino acid | As shown in SEQ ID NO. 3 |
| sequence of MMP | |
| restriction enzyme | |
| cutting sites | |
| Amino acid | As showN in SEQ ID NO. 4 |
| sequence of the | |
| therapeutic | |
| polypeptide | |

1.2. Construction of the Recombinant Genetically Engineered Bacteria

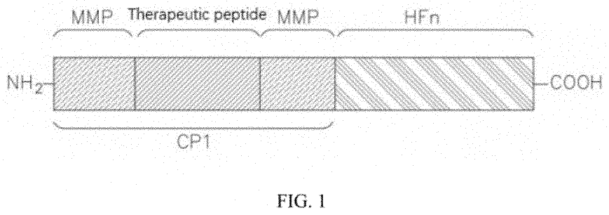

The pET-28a-HFn-CP1 and pET-28a-HFn correctly constructed above (as a control, not as shown in FIG. 1 or FIG. 2) were transformed into E. coli Rosetta (DE3) competent cells, respectively. 7 single colonies were respectively picked overnight and shaken; the 1% bacterial solution was transferred onto IPTG at 4 h and 0.5 mM for 6 h; an empty vector pET-28a served as a blank control and a recombinant plasmid without induced expression served as a negative control; the SDS-FAGE protein was subjected to electrophoresis detection to pick out the bacteria with high expression quantity; then 50% glycerol was added and preserved at −80° C., namely, obtaining the recombinant genetically engineered bacteria.

1.3. Induced Expression and Purification of the Recombinant Proteins HFn-CP1 and HFn

The recombinant genetically engineered bacteria were subjected to streak culture on a plate containing Kana+resistance, and monoclonal antibodies were picked up and inoculated onto a 5 mL. LB liquid medium containing 50 μg/ml Kana +, and cultured for 12-16 h at 37° C. and 180 r/min. The bacterial solution was transferred into a 500 mL LB liquid medium according to an inoculum size of 1%, and added with kanamycin antibiotics, then subjected to shaking culture at 37° C. and 180 rpm until OD600 of the bacterial solution was 0.4-0.6; an inducer IPTG was added to a final concentration of 1 mmol/L; and the bacterial solution was continuously subjected to induced culture at 20° C. for 16 h, and then centrifuged for 8 min at 4° C. at 8000 rpm, then bacterial cells were collected. The pre-cooled ferritin was assembled with a buffer to clean the bacterial cells once, and centrifugation was performed to discard supernatant, and bacterial cells were collected.

The collected bacteria cells were resuspended in a Binding buffer containing 5 mM imidazole, disrupted with a sonicator for 40 min in an ice bath, then centrifuged for 40 min at 4° C., 1000×g to remove precipitates.

The supernatant was taken as an initial protein sample, and subjected to nickel ion-chelating chromatography gradient elution; the imidazole had a concentration gradient of 10 mM, 20 mM, 50 mM, 100 mM and 500 mM to obtain the purified recombinant protein suspension HFn-CP1 and the control protein HPn.

1.4. Western Blotting Assay of the Recombinant Protein

The purified recombinant protein sample HFn-CP1 was subjected to SDS-PACE protein get electrophoresis; the protein sample on the gel was transferred onto a PVDF membrane by a wet transfer method; the PVDF membrane was removed and washed for 3 limes with a PBST cleaning solution and blocked with 3% BSA for 1.5 h. At the end of the blocking, the protein was washed for 3 times with a PBST cleaning solution, and then incubated for 2 h by murine ferritin polyclonal antibody diluted by 500 folds as a primary antibody. The protein was cleaned with PBST for 3 times, and diluted by 1000 folds and incubated for 2 h with a HRP-labeled goat-anti-mouse IgG, and cleaned for 3 times with PBST, then developed with a BeyoECL plus AB mixed solution and observed.

2. Preparation of HPn-CP1 (Adr+QD)

2.1. Loading of Quantum Dots and Adriamycin

Sample whose target bands were the dominant proteins were firstly healed at 60° C. for 10 min, then centrifuged at 4° C. and 10000 rpm to remove the heat-labile protein; and the protein of interest (HFn-CP1 and control protein HFn) were further purified, followed by the packaging of quantum dots.

It has been reported in the literature that ferritin nanocages will be depolymerized upon denaturation at high (8 M) concentration of urea, and will be self-assembled after the concentration of urea was diluted slowly. This property may help the ferritin nanocages load a medicament and other optical particles.

The specific steps were as follows: 1 mg/mL HFn-CP1 was dissolved into 8 M urea and gently vortexed for 30 min at room temperature to ensure complete dissociation; afterwards, adriamycin (abbreviated as Adr) at a final concentration of 1 mg/mL and quantum dots (abbreviated at QD, Suzhou NIROPTICS Technology Co. Ltd.) at a final concentration of 10 μg/mL were added to the solution. After being incubated for 10 min in the dark, the mixture was transferred to a dialysis bag (molecular weight cut-off of 3000 Da) such that the HFn protein cage was reassembled slowly in Adr containing 1 mg/mL at 4° C. for 4 h each directed to a gradient concentration of urea buffer solution (7, 5, 3, 2, 1 and 0 M dialysis). Finally, the obtained solution was dialyzed overnight with saline to remove free Adr to obtain loaded HFn-CP1(Adr+QD) and HFn(Adr+QD) nanoparticles, where the concentrations of Adr and quantum dots were determined by UV absorption.

2.2. Purification of Loaded Nanoparticles

The dialyzed mixture was further purified by sucrose density gradient centrifugation. The steps are as follows:

Preparation of sucrose density gradient: 50 g sucrose were weighed and dissolved into 50 mL ferritin to be assembled into to a buffer solution, then prepared a sucrose mother solution with a concentration of 50% (W/W), the ferritin was assembled with the buffer and diluted into sucrose gradient solutions having concentration of 10%, 15%, 20%, 25%, 30%, 35%, 40%, 45% and 50%; transparent centrifuge tubes dedicated for Beckman SW41 rotor were divided into 10 parts with equal volume; the sucrose gradient solutions were added to the centrifuge tubes by equal volume according to the order of concentration from high to low; disturbance should be avoided between different concentrations of the solutions during the adding process. The prepared sucrose gradients were placed into a 4° C. refrigerator overnight to form continuous gradients.

The protein samples were added to the top of the sucrose density gradients to reserve space, and centrifuged at 4° C. and 38000 rpm for 4 h; after the centrifugation, the protein of interest solution was taken out according to the colors of the quantum dots. The protein of interest was dialyzed in the ferritin-assembled buffer to remove sucrose, and the protein was concentrated using an ultrafiltration tube as required, namely, obtaining the purified ferritin nanoparticles loaded with quantum dots and adriamycin.

The purified protein was subjected to concentration determination using a BCA protein concentration determination kit. The purified protein was sub-packaged and cryopreserved in a −80° C. refrigerator to avoid repeated freezing and thawing.

3. Characterization of HFn-CP1(Adr+QD)

3.1 Dynamic Light Scattering and Zeta Potential Characterization

The sample were added to a Size micro-sample cell or Zeta potential sample cell; nano-particle size and parameters of a surface potential analyzer (the measurement temperature was set as 37° C., and the measurement angle was 173°) were set, thus measuring the particle size distribution or Zeta potential of the particles.

3.2 Characterization of Transmission Electron Microscopy (TEM)

Tweezers were used to carefully clamp the edge of the TEM carbon-supported membrane copper screen and take it out; the protein sample was diluted to a concentration of about 0.1 mg/mL, and a sealing film on ice fur pre-cooling; 10 μL sample was dropped onto the scaling film, and the carbon-supported surface of the copper screen was adsorbed on the liquid drop for about 1 min; the edge liquid drops were absorbed by a qualitative filter paper, and the surface of the copper screen adsorbing the sample was subjected to negative staining on a 2% phosphotungstic acid solution for 5 s; the edge liquid drops were absorbed by a qualitative filter paper carefully, and the surface of the copper screen adsorbing the sample was put on the filter paper sheet in a face-up way for ventilation and drying for a night; transmission electron microscopy and photographing were taken on the next day.

4. Cell Experiment

Cell thawing: MDA-MB-468 cells (purchased from Guangzhou Cellcook Biotech CO, Ltd., product number: CC0309) cryopreserved at −80° C. were rapidly resuscitated in a thermostatic water bath at 37° C. The cell suspension in the cryopreserved tube was transferred to centrifuge tubes, and added to 2 mL L-15 complete medium containing 10% FBS, and centrifuged at 1000 rpm for 4 min: then supernatant was discarded. The cells were resuspended in a fresh complete medium and then transferred to a T-25 cell culture flask.

Cell culture: human breast cancer cells MDA-MB-468 were cultured in L-15 complete medium containing 10% PBS in a 37° C. thermostatic incubator. The cells were adherent.

Cell passage: 2-3 days later after being cultured, the cells may be subcultured when the degree of MDA-MB-468 cell convergence was up to 80% and the cell morphology was observed to be normal. After the culture medium in the culture flask was absorbed and discarded, the cells were cleaned with 2 mL PBS buffer; after the PBS was absorbed and discarded, 1 mL of 0.25% pancreatin was added to digest the cells. The cells were placed in an incubator at 37° C. for 3 min; 2 mL complete culture medium was added to the flask to terminate the digestion after observing that the cells were completely digested under an inverted microscope. The cell suspension was pipetted into a 15 mL centrifuge tube and centrifuged for 5 min at 1000 rpm. The supernatant was discarded, and the cell mass was resuspended with a certain amount of complete culture medium, and then subcultured in a proportion of 1:2.

Cell cryopreservation: a cell cryopreservation solution (10% FBS+10% DMSO) +80% culture medium was prepared: the process was the same as that before resuspension of the subcultured cells except the cell mass was resuspended with the cryopreservation solution and transferred into a cryopreservation tube. Finally, the cryopreservation tube was put to a gradient-cooling cryopreservation box and preserved at −80° C.

Cell treatment: when the degree of MDA-MB-468 cell convergence was up to 80%, the prepared adriamycin and quantum dots were prepared into 1 mg/mL stock solutions; HFn(QD) (quantum dots were loaded with ferritin only), HFn-CP1(QD) (quantum dots were loaded with the recombinant ferritin only), HFn(Adr+QD), HFn-CP1(Adr+QD) were concentrated to 0.1 mg/mL using ultrafiltration tubes. The cells were diluted by a phenol red-free RPMI 1640 medium. The second group of MTT experiment was a gradient concentration inhibition experiment of HFn-CP1(Adr+QD) nanoparticles. In the third group of MTT experiment, to verify the therapeutic effects of the cancer therapeutic polypeptide and ferritin nanocages, the variable Adr was controlled the same to determine the viability of breast cancer cells.

10 μL Adr were taken respectively; one was diluted with PBS to a 125 μg/mL Adr solution, and another one was diluted with HFn-CP1 nanoparticles to a 125 μg/mL Adr and 100 μg/mL HFn-CP1 solution, then kept for 4 h at 60° C. such that the hydrophilic drug channel of the ferritin nanocages was opened to allow Adr to enter into the nanocages. After being filtered and sterilized by a 0.22 μm filter membrane, the cells were subjected to MTT assay.

Determination of Cell Viability by MTT Assay:

Cell treatment: after the MDA-MB-468 cells were digested with 0.25% pancreatin, the cells were counted by a blood counting plate; the cell suspension was diluted according to the amount of 8000 cells per well of a 96-well plate; the diluted cell suspension was added to the 96-well plate by 200 μL per well; then the 96-well plate was put back to the 5% CO₂ incubator at 37° C. for continuous culture overnight, after cell attachment again, subsequent experiments were performed.

Medicament treatment: after the cells converged to 80%, the culture medium in the 96-well plate was sucked out; and each well was gently washed once with PBS buffer. In the first group of MTT experiment, 0.1 mg/mL HFn(QD), HFn-CP1(QD), HFn(Adr+QD), HFn-CP1(Adr+QD) and the corresponding concentrations of QD and Adr were added respectively; meanwhile, the blank control group was set. In the second group of MTT experiment, HFn-CP1(Adr+QD) was respectively diluted to 0.01, 0.1, 1, 10, 100 μg/mL, then added to 96-well plates. In the third group of MTT experiment, the Adr solution added with HFn-CP1 and Adr solution with the same concentration were diluted to 0.62, 1.25 and 2.5 μg/mL of Adr, respectively. Six replicate wells were set for each concentration, and 100 μL of prepared drug solution was added to each well. Meanwhile, zeroing wells were set (cells were not inoculated, but the subsequent operation was the same as that in the control group). After administration, the cells were put back to the incubator for continuous culture tor 24 h.

MIT reaction: 24 h later, the culture medium in each well of the 96-well plate was carefully sucked and discard, and gently washed with PBS buffer; fresh phenol red-free RPMI 1640 culture medium and 10 μL prepared MTT solution (5 mg/mL, dissolved in PBS) were added to each well, and then continuously cultured in the 5% CO₂ incubator at 37° C. for 4 h.

4 h later, the culture medium in the 96-well plate was sucked and discarded; 100 μL DMSO was added to each well, and gently shaken to completely dissolve the crystal; the whole process should be kept in the dark. The completely dissolved 96-well plate was put on a microplate reader, and parameters were set to determine the absorbance of each well at 570 nm.

Cell viability calculation: cell viability %=(absorbance in the experimental group-absorbance in the zeroing group)/(absorbance in the control group-absorbance in the zeroing group)*100%

5. Data Statistics and Analysis

The experimental data are represented by mean±standard deviation, and the data are tested for significance of difference by T-test. When P>0.05, the difference is not significant; P<0.05 (*) indicates a statistic difference; P<0.01 (**) indicates a statistically significant difference and P<0.001 (***) indicates an extremely statistically significant difference.

II. Results

1. Expression and Purification of the Protein of Interest

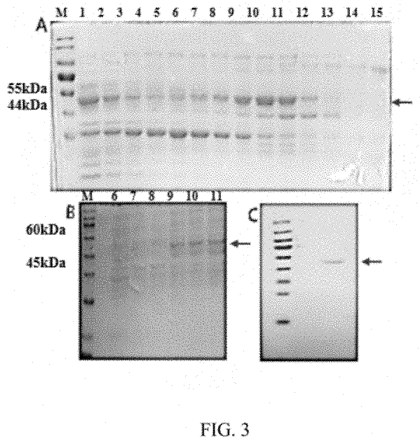

Purification of the HFn-CP1 protein; the sonicated protein supernatant was added to a nickel column equilibrated with a binding buffer, and after loading the samples twice in cycles, the nickel column was eluted with two column volumes of wash buffer and elution buffer, and the samples were collected to achieve the primary purification effect of the protein of interest. The collected samples of the protein of interest were detected by SDS-PAGE; obvious bands may be seen between 45 kDa and 55 kDa. The eluted samples of the protein of interest were heated at 60° C. for 10 min, and centrifuged for 30 min at 10000 rpm and 4° C. and the protein supernatant was taken to obtain the relatively purer protein samples, and Western Blot was used for verification, as shown in FIG. 3.

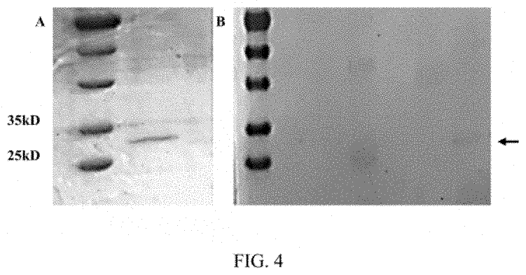

Purification of the HFn protein: purification was conveniently performed by nickel-column affinity chromatography; in this example, a segment of 6× his tags was added on a N-terminal of the wild-type ferritin. The protein was subjected to the same elution process by the nickel column, heated at 60° C. and centrifuged at 4° C. to obtain a relatively purer wild-type ferritin. As shown in FIG. 4. SDS-PAGE and Western Blot results show that the protein of interest HFn is between 25 kDa and 35 kDa.

2. Loading of Quantum Dots and Adriamycin

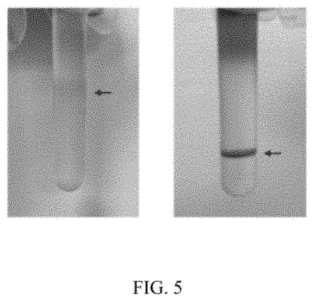

After ferritin was depolymerized by 8M urea, quantum dots and adriamycin were added and gently vortexed for 30 min; the urea was gradually diluted such that ferritin was gradually polymerized to complete the packaging of the quantum dots and adriamycin. To separate free quantum dots, adriamycin and the non-successfully packaged ferritin. In the example, sucrose gradient centrifugation was utilized for separation, after centrifugation, protein bands with a distinct color prompt, as shown in FIG. 5. In FIG. 5, the quantum dots are yellow and the adriamycin is red. The left one shows the HFn-CP1 packaged with quantum dots only, and the right one shows HFn-CP1 packaged with both quantum dots and adriamycin. The results show a yellow protein band on the left and a dark red band on the right, indicating that the loading of quantum dots is completed by ferritin on the left and the loading of quantum dots and adriamycin is completed on the right.

3. Characterization of Nanoparticles

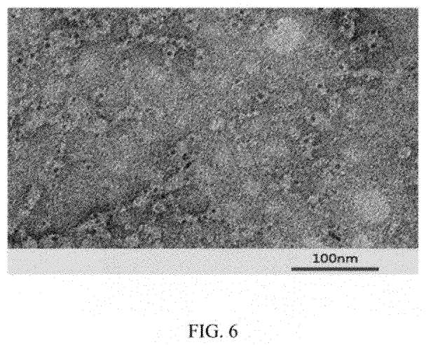

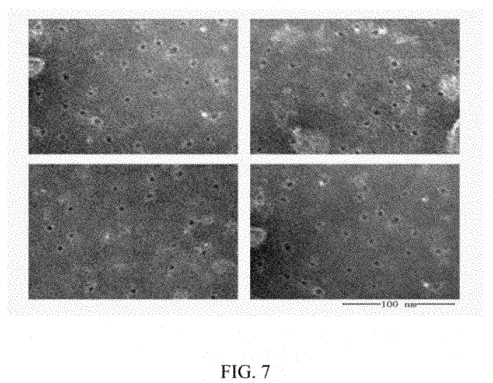

To verify she structure of the nanoparticle which has completed loading, in this example, the HFn-CP1 protein was characterized using transmission electron microscopy (TEM). The HFn-CP1 protein was adsorbed on the surface of the copper screen, and then stained with a negative staining solution. After the excessive liquid was blotted up with a filter paper, the copper screen was air-dried and prepared into electron microscope samples. FIG. 6 shows that HFn(QD) is a cage-like structure, black represents quantum dots, and the quantum dots are surrounded by a circle of white protein, and the nanoparticle is about 10 nm. As shown in FIG. 7, the structures of HFn-CP1(QD) and HFn(QD) are basically the same, which indicates that the modified polypeptide added in the nanoparticle HFn-CP1(QD) will not affect the cage-like structure of ferritin, and proves that the quantum dots have been successfully wrapped by ferritin cages.

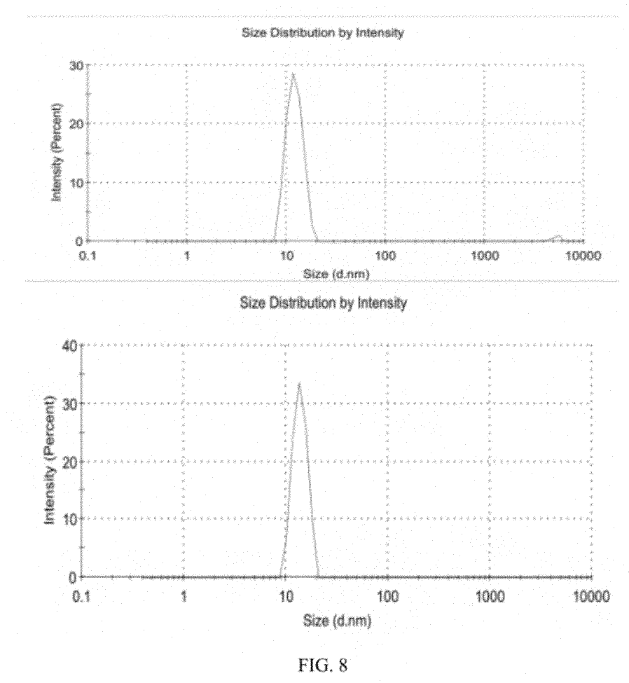

The distribution of the nanoparticles is characterized by dynamic light scattering (DLS), as shown in the lower graph of FIG. 8 below. The HFn-CP1(QD) nanoparticles have a particle size of 13.88±0.69 nm and a zeta potential of 12.63±1.05 mV. As shown in the upper graph of FIG. 8, the wild-type ferritin HFn(QD) has a particle size of 12.26*1.62 nm and a zeta potential of 11.58±1.06 mV. Therefore, the results show that the outer diameter of HFn-CP1(QD) nanoparticles is slightly greater than that of the wild-type ferritin HFn(QD), which indirectly proves that the surface of HFn-CP1(QD) nanocage is modified.

4. Results of the Cell Experiment

To verify the inhibitory effect of HFn-CP1(Adr+QD) nanoparticles on breast cancer cells, a series of MTT experiments were also performed in the examples of the present disclosure to confirm the effectiveness of the HFn-CP1(Adr+QD) nanoparticles. In the examples, QD, ADr, HFn(QD), HFn-CP1(QD), HFn(Adr-QD), and HFn-CP1(Adr+QD) were respectively added to 96-well plates overspread with MDA-MB-468 breast cancer cells under the same conditions, and treated for 48 h, then added with MTT for treatment for 4 h; OD value of each well was measured.

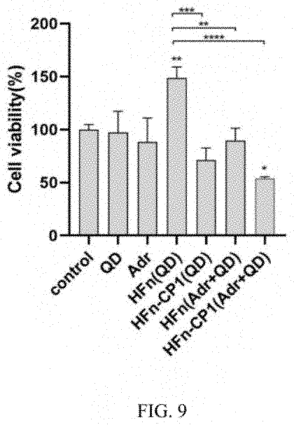

The results are shown in FIG. 9, QD has little effect on cell viability, while HFn(QD) accelerates the growth of cancer cells. Comparison of the results of HFn(QD) and HFn-CP1(QD) shows that the therapeutic peptide CP1 wrapped by ferritin may be released and also has a significant therapeutic effect. HFn-CP1(Adr+QD) has the strongest inhibitory effect on the viability of breast cancer cells relative to other groups. In general, both the small peptide for cancer therapy and adriamycin may play therapeutic effects, and produce inhibiting effects on the viability of breast cancer cells.

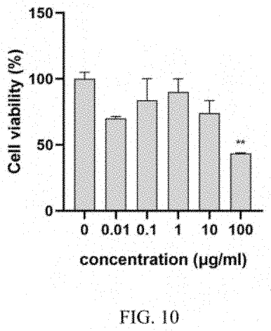

To further confirm the impact of HFn-CP1(Adr+QD) nanoparticles on the viability of breast cancer cells, in the examples, the culture media added with different concentration gradients of HFn-CP1(Adr+QD) nanoparticles were added to 96-well plates overspread with MDA-MB-468 breast cancer cells, and treated for 48 h, then added with MTT for further treatment for 4 h; OD value of each well was measured. The results are shown in FIG. 10. 100 μg/mL of HFn-CP1(Adr+QD) nanoparticles has a significant inhibitory effect on cell viability; the inhibition on cell viability is concentration dependent between 1 and 100 μg/mL.

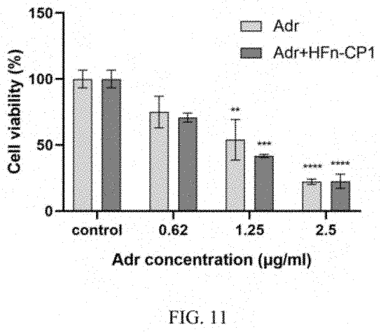

To verify the therapeutic effects of the cancer therapeutic polypeptide and ferritin nanocages, the variable Adr was controlled the same in this example to determine the viability of breast cancer cells. Same volume of Adr stock solutions were taken; one was diluted by PBS, and another one was diluted by adding 100 μg/mL HFn-CP1 nanoparticle solution (Adr and QD were not loaded); similarly, with the Adr concentration was diluted to 125 μg mL, heated at 60° C. such that Adr was allowed to enter into the nanocages. After being filtered and sterilized by a 0.22 filter membrane, the two samples were subjected to the determination on the viability of breast cancer cells. The results are shown in FIG. 11. At the same concentration of 1.25 μg/mL of Adr, the sample added with HFn-CP1 nanoparticles has more significant cell inhibition effect, which indicates that the therapeutic polypeptide and ferritin nanocages may enhance the inhibition effect of Adr on cancer cells.

In conclusion, recombinant ferritin merged with a therapeutic polypeptide for cancer cells is constructed in the examples of the present, disclosure, and unique nanoparticles of recombinant ferritin loaded with quantum dots and adriamycin are designed as a drug delivery system; moreover, the efficacy thereof has been proved by cell experiments. The results indicate that the recombinant ferritin can release the therapeutic peptide in cells better, and the adriamycin loaded can also be precisely released to achieve targeted therapy. Moreover, as a drug delivery system, the multi-functional nanoparticle may achieve the optical imaging of tumor cells by loading quantum dots, thereby playing a role in cancer diagnosis and treatment.

What is described above are merely preferred detailed embodiments of the present disclosure, but the protection scope of the present disclosure is not limited thereto. Any change or replacement capable of being readily envisaged by a person skilled in the art within the technical scope disclosed herein may fall within the protection scope of the present disclosure.

### SEQUENCE LISTING

<110> Wuhan University
<120> MULTI-FUNCTIONAL NANOPARTICLE TARGETED TO BREAST CANCER, PREPARATION METHOD AND USE THEREOF
<141> Jun. 16, 2021
<160> 4
<170> SIPOSequenceListing 1.0
<210> 1
<211> 18
<212> DNA
<213> Artificial Sequence
<400> 1

| | |
|---|---|
| ccgctgggtc tgtgggca | 18 |

<210> 2
<211> 72
<212> DNA
<213> Artificial Sequence
<400> 2

| | |
|---|---|
| tatggtcgtaaaaagcgtcgtcagcgtcgtcgtcgtaaacgtcgtaaagcaaccgatgaa | 60 |
| atgattccgt tt | 72 |

<210> 3
<211> 6
<212> PRT
<213> Artificial Sequence
<400> 3

Pro Leu Gly Leu Trp Ala
1 5

1 5
<210> 4
<211> 14
<212> PRT
<213> Artificial Sequence
<400> 4

Thr Ala ThrAsn Leu Ser Ala Thr Asp Glu Met Ile Pro Phe
1 5 10

What is claimed:

1. A multi-functional nanoparticle targeted to breast cancer, comprising a targeting carrier and a medicament loaded on the targeting carrier, wherein the targeting carrier is made from recombinant ferritin.

2. The multi-functional nanoparticle according to claim 1, wherein the recombinant ferritin has a primary structure formed by linking a therapeutic polypeptide, MMP restriction enzyme cutting sites to ferritin.

3. The multi-functional nanoparticle according to claim 2, wherein the MMP restriction enzyme cutting sites are linked at both ends of the therapeutic polypeptide.

4. The multi-functional nanoparticle according to claim 3, wherein the therapeutic polypeptide is a Wnt/β-catenin signaling inhibitor which is capable of preventing β-catenin from binding to LEF-1 in a nuclear region of human breast cancer cells, thereby accelerating the apoptosis of cancer cells, and reducing the growth and motility of cancer cells.

5. The multi-functional nanoparticle according to claim 1, wherein the medicament comprises quantum dots and adriamycin.

6. A method tor preparing the multi-functional nanoparticle according to claim 1, comprising steps of preparing recombinant ferritin and loading the medicament, wherein the step of preparing the recombinant ferritin comprises:
constructing a genetically engineered strain expressing the recombinant ferritin;
subjecting the genetically engineered strain to plate culture and liquid induction culture to obtain a bacterial solution containing the recombinant ferritin;
collecting bacterial cells in the bacterial solution, crushing and purifying to obtain the recombinant ferritin.

7. The preparation method according to claim 6, wherein the construction process of the genetically engineered strain comprises: constructing an expression vector pET28a-HFn-CP1, transforming the expression vector into E. coli Rosetta (DE3) competent cells, performing streaking culture on a kana+plate, and picking a positive monoclonal colony as the genetically engineered strain;
wherein the expression vector comprises nucleotide sequences for expressing the MMP restriction enzyme cutting sites, the therapeutic polypeptide and the ferritin respectively; the nucleotide sequence for expressing the MMP restriction enzyme cutting sites is shown in SEQ ID NO. 1 and the nucleotide sequence for expressing the therapeutic polypeptide is shown in SEQ ID NO. 2.

8. The preparation method according to claim 6, wherein the step of loading the medicament comprises:
adding quantum dots and an adriamycin solution to a urea solution of the recombinant ferritin for mixing, performing incubation in the dark, then dialyzing the same in a urea buffer solution to obtain a concentrate;
subjecting the concentrate to sucrose density gradient centrifugation, thus obtaining the finally purified multi-functional nanoparticle.

9. The preparation method according to claim 8, wherein the sucrose solution has concentration gradients of 10 w/w %, 15 w/w %, 20 w/w %, 25 w/w %, 30 w/w %, 35 w/w %, 40 w/w %, 45 w/w % and 50%, respectively.
